(19) United States
(12) Patent Application Publication
(10) Pub. No.: US 2025/0044220 A1
(43) Pub. Date: Feb. 6, 2025

(54) APPARATUS AND METHOD FOR FLUID ANALYTE DETECTION USING RING RESONATOR BASED SPECTROSCOPY

(71) Applicant: UNIVERSITY OF SOUTHERN CALIFORNIA, Los Angeles, CA (US)

(72) Inventors: Ajey P. JACOB, Los Angeles, CA (US); Akhilesh JAISWAL, Los Angeles, CA (US); Ramesh KUDALIPPALLIYALIL, Los Angeles, CA (US); Sujith CHANDRAN, Los Angeles, CA (US)

(21) Appl. No.: 18/923,395

(22) Filed: Oct. 22, 2024

Related U.S. Application Data
(63) Continuation of application No. PCT/US2023/019633, filed on Apr. 24, 2023.
(60) Provisional application No. 63/333,801, filed on Apr. 22, 2022.

(51) Int. Cl. G01N 21/25; G01N 21/27; G01N 33/569; G01N 33/68

(52) U.S. Cl. CPC G01N 21/255; G01N 21/272; G01N 33/56983; G01N 33/6848; G01N 2333/165

(57) ABSTRACT

Provided is a device, device for analyte detection, comprising: a first waveguide; a sensor ring resonator, optically coupled to the first waveguide, wherein the sensor ring resonator is sensitive to an analyte; multiple filter ring resonators optically coupled to the sensor ring resonator, one or more detectors, wherein each of the multiple filter ring resonators is optically coupled to at least one of the one or more detectors; and at least a first microfluidic channel, wherein the first microfluidic channel configured to fluidically deliver an analyte to the sensor ring resonator.

### CROSS-REFERENCE TO RELATED APPLICATIONS

This application claims benefit of U.S. Provisional Patent Application 63/333,801 titled APPARATUS AND METHOD FOR VIRAL DETECTION USING RING RESONATOR BASED SPECTROSCOPY, filed 22-4-2022. The entire contents of each aforementioned patent filing are hereby incorporated by reference.

### BACKGROUND

### 1. Field

The present disclosure relates generally to spectrometry and analyte detection.

### 2. Description of the Related Art

Available COVID 19 and other viral detection methods may be time-consuming, expensive, and require trained operation and interpretation. In some application, integrated photonic sensors (including arrayed waveguide on-chip spectrometry, echelle diffraction grating based on-chip spectrometry, etc.) may be used for COVID 19 or other viral detection, but may suffer from temperature dependency and fabrication sensitivity and therefore require the use of extrinsic spectral calibration devices. None of which is to suggest that any techniques suffering from these issues is disclaimed or that any other discussion of engineering tradeoffs herein constitutes a disclaimer.

### SUMMARY

The following is a non-exhaustive listing of some aspects of the present techniques. These and other aspects are described in the following disclosure.

Some aspects include a selectively-sensing photonic microfluidic optical ring resonator-based integrated chip architecture with an on-chip spectrometer consisting of coupled ring resonator filters and integrated photodetector arrays.

Some aspects include an integrated chip architecture which reduces fabrication-induced performance variation and thermal sensitivity.

Some aspects include a sensor, including: a sensing ring resonator functionalized to be sensitive to an analyte optically coupled to multiple ring resonators operating as filters, with one or more photodetectors optically coupled to each of the multiple ring resonators, operating as a spectrometer.

Some aspects include an integrated optical source.

Some aspects include a microfluidic system which delivers an analyte delivery fluid, which may or may not contain analyte, to the sensing ring resonator.

Some aspects include a microfluidic system which also delivers analyte delivery fluid, which may or may not contain analyte, to the multiple ring resonators.

Some aspects include one or more additional filters, which may be additional ring resonator filters, arrayed waveguides, arrayed waveguide gratings, etc.

Some aspects include integrated passive photonics.

Some aspects include integrated active photonics.

Some aspects include an integrated microfluidic layer.

Some aspects include a microfluidic layer applied to an integrated photonic layer.

Some aspects include ring resonators which are slot ring resonators, subwavelength grating ring resonators, etc.

Some aspects include multiple of the above-described apparatus, including in series, in parallel, etc.

Some aspects include an array of multiple of the above-described apparatus.

Some aspects include a method of fabricating the above-described apparatus.

Some aspects include a method of operating the above-described apparatus.

### BRIEF DESCRIPTION OF THE DRAWINGS

The above-mentioned aspects and other aspects of the present techniques will be better understood when the present application is read in view of the following figures in which like numbers indicate similar or identical elements:

FIG. 1A is a schematic representation of a ring resonator based photonic circuit with split fluid channel for sensing, according to some embodiments.

FIG. 1B is graph depicting example waveguide sensitivity to transverse electric (TE) and transverse magnetic (TM) modes, according to some embodiments.

FIG. 2A is a schematic representation of a ring resonator based photonic circuit with a single fluid channel for sensing, according to some embodiments.

FIG. 2B is a schematic representation of a ring resonator based photonic circuit for sensing integrated in a fluid analyzer, according to some embodiments.

FIG. 3 is a schematic representation of a cross-section of a ring resonator based photonic circuit, according to some embodiments.

FIGS. 4A-4B are graphs depicting a response of a ring resonator based photonic circuit to exposure to a virus particle, according to some embodiments.

FIGS. 5A-5B are graphs depicting a response of a ring resonator based photonic circuit to exposure to multiple virus particles, according to some embodiments.

FIGS. 6A-6C are graphs depicting responses of a ring resonator based photonic circuit to different virus exposure levels, according to some embodiments.

FIGS. 7A-7D are graphs depicting responses of a ring resonator based photonic circuit for different fabrication conditions, according to some embodiments.

FIG. 8 is a schematic representation of a series of ring resonator based photonic circuits, according to some embodiments.

FIG. 9 illustrates an example computing system with a ring resonator photonic circuit, according to some embodiments.

While the present techniques are susceptible to various modifications and alternative forms, specific embodiments thereof are shown by way of example in the drawings and will herein be described in detail. The drawings may not be to scale. It should be understood, however, that the drawings and detailed description thereto are not intended to limit the present techniques to the particular form disclosed, but to the contrary, the intention is to cover all modifications, equivalents, and alternatives falling within the spirit and scope of the present techniques as defined by the appended claims.

### DETAILED DESCRIPTION OF CERTAIN EMBODIMENTS

To mitigate the problems described herein, the inventors had to both invent solutions and, in some cases just as importantly, recognize problems overlooked (or not yet foreseen) by others in the image detection and image processing. Indeed, the inventors wish to emphasize the difficulty of recognizing those problems that are nascent and will become much more apparent in the future should trends in industry continue as the inventors expect. Further, because multiple problems are addressed, it should be understood that some embodiments are problem-specific, and not all embodiments address every problem with traditional systems described herein or provide every benefit described herein. That said, improvements that solve various permutations of these problems are described below.

The description that follows includes example systems, methods, techniques, and operation flows that illustrate aspects of the disclosure. However, the disclosure may be practiced without these specific details. For example, this disclosure refers to specific types of resonators (e.g., ring resonators, optical ring resonators, disk resonators, etc.), specific types of waveguides (e.g., strip waveguide, slot waveguide, etc.) specific types of biological material (e.g., viruses, viral proteins, bacteria, biomarkers, etc.), and specific types of integration (e.g., heterogeneous, homogeneous, etc.) in illustrative examples. Aspects of this disclosure may instead be practiced with other or additional types of resonators, waveguides and photonic materials, biological materials, and integration. Additionally, aspects of this disclosure may be practiced with other types of optical sources (e.g., lasers, broadband sources, photodiodes, integrated optical sources) and detectors (e.g., optical detector, photodetector, photodiode, photoconductor, etc.). Further, well-known structures, components, instruction instances, protocols, and techniques have not been shown in detail to not obfuscate the description.

COVID 19 viral detection, as well as other viral and biological agent detection, may be useful as a point-of-care indicator (e.g., on the patient scale) and as a pandemic tracking and mitigation method (e.g., on the population scale). For case of use, a viral detector that is sufficiently portable (for case of distribution), cheap (or relatively cheaper than benchtop analysis), robust (to storage and testing environments), etc. may be desired. Currently, COVID 19 viral detection—and viral load detection—methods may be time-consuming, expensive, imprecise, or require expert operation or interpretation (e.g., electrophoresis, polymerase chain reaction (PCR) analysis, etc.).

Photonic sensors may be used to provide spectroscopic analysis for viral detection but may experience temperature dependence, which may require temperature stabilization equipment, temperature effect removal devices or techniques, etc., and may be sensitive to fabrication variations, which may require individual device calibration, device geometry measurement, etc. Previous photonic viral detection schemes tended to rely on external spectrometry for these (and other) reasons, which may increase lag times in detection (when samples are analyzed with bench-top quality external devices) and costs and decrease throughput.

In some embodiments, by combining multiple ring resonators with microfluidic channels, a selective-sensitive optical ring resonator sensor may be fabricated. In some embodiments, the microfluidic channels may operate to deliver an analyte (e.g., one or more virus or other substance to which the sensor is sensitive), such as via an analyte delivery fluid, to a functionalized ring resonator (e.g., sensor ring resonator). In some embodiments, adsorption, absorption, adhesion, and other native or added functionality may be used to alter the characteristics (e.g., optical path length) of a ring resonator (e.g., sensor ring resonator) when exposed to an analyte. In some embodiments, an on-chip spectrometer made up of coupled ring resonators (e.g., filter ring resonators) may be fabricated, which may reduce output variation caused by fabrication, thermal variation, or the like. In some embodiments, an integrated chip may also contain integrated photodetectors, including one or more photodetector array, one or more integrated optical source (e.g., light emitting diode, photodiode, etc.), and one or more filters (e.g., optical filter)—which may be resonator filters. In some embodiments, an integrated chip, in which multiple photonic components are fabricated in situ and experience similar conditions, may be expected to improve performance by reducing the effects of thermal variation, fabrication variation, etc. Additionally, in some embodiments, design parameters may be adjusted to reduce thermal and fabrication variation effects on sensor output and reduce post-processing computational needs. It is expected that some embodiments of the photonic device herein described may be deployed as a point of care sensor to provide patient level COVID 19 detection (or patient level detection of another virus or biological agent) that is robust to temperature and fabrication sensitivities.

It is expected that aspects herein described for COVID 19 detection may instead or additionally apply to detection of other viruses, other airborne pathogens, such as bacteria, fungus, etc., and non-infectious airborne particles, such as allergens (e.g., pollen), particulates, volatile organic compounds (VOCs), etc., including pathogens and particles which are substantially larger or smaller than viruses. In some embodiments, it is expected that aspects described for COVID 19 detection may instead or additionally apply to detection of viruses, pathogens, or particles in one or more liquid, or pathogens or particles which are suspended or dissolved in one or more gas, including viruses, pathogens, or particles which are introduced to a test gas or liquid by a pre-treatment apparatus or stage. Herein, fluid should be understood to encompass liquids, fluids, and other substances which can be caused to flow.

In some embodiments, an integrated chip may combine microfluidic, spectrometry, and analytical circuitry. Microfluidic circuitry may include one or more pumps-which may include a fluid pump (e.g., an air pump, a liquid pump, a pump which delivers an analyte (e.g., in a first medium) into an analyte deliver fluid, etc.) and which may circulate or otherwise deliver an analyte delivery fluid to the sensing ring resonator (and, in some embodiments, to the filter ring resonators). Microfluid circuitry (e.g., channels, mixing chambers, etc.), in some embodiments, may be in plane with the photonic circuitry and may be integrated with the photonic circuitry—e.g., can be fabricated of SiN, Si, Ge, SiGe, III-V materials etc. which may make up material of active and passive photonic elements. The microfluidic circuitry, in some embodiments, may be fabricated from heterogeneous material (e.g., polymers). The microfluidic circuitry may be applied as a top layer or additional layer on top of the photonic circuitry. The microfluidic circuitry (e.g., microfluidic channels), in some embodiments, may be open to a chamber or atmosphere, or may comprise tunnels or channels otherwise surrounded by sidewalls, ceilings, floors, etc. The microfluidic circuitry, in some embodiments, may operate in plane with the photonic circuitry (e.g., parallel to the plane of the ring resonators) and may also operate perpendicular or at another angle to the plane of the chip. The microfluidic circuitry, in some embodiments, may also include one or more ports for entrance and exit of analyte containing fluid, analyte containing air, analyte delivery fluid, air, etc. The sensor ring resonator, in some embodiments, may occupy the same microfluidic channel as the ring resonators of the optical filter (e.g., filter ring resonators), such as downstream or upstream from the sensor ring resonator. The ring resonators of the optical filter and the sensor ring resonators, in some embodiments, may occupy different microfluidic channels, including microfluidic channels with a common source (e.g., of analyte deliver fluid) or common drain. The ring resonators of the optical filter, in some embodiments, may occupy a microfluidic channel which is not fluidically coupled to a microfluidic channel of the sensor ring resonator, or even a microfluidic reservoir or other non-communicative microfluidic channel.

In some embodiments, analytical circuitry may operate on output of the photonic circuit, including upon output of the filter ring resonators. In some embodiments, analytical circuitry may include heterogeneously integrated circuitry, such as an application specific integrated chip (ASIC) communicatively coupled to the output(s) of a spectrometer (e.g., of detector(s) coupled to the filter ring resonators). The analytical circuitry may be electrically or optically connected to the one or more photonic device (e.g., to one or more of the sensor ring resonators, the filter ring resonators, an optical source, detector(s), waveguides, etc.). The analytical circuitry may be fabricated in plane with the one or more photonic devices, including in a layer or region containing active photonics or passive photonics. The analytical circuitry may include one or more processors, memory or other data storage, communication circuitry including wireless communication circuitry, display circuitry (e.g., digital read output circuitry, analog read output circuitry, etc.), etc. The analytical circuitry may include one or more sensors (e.g., in addition to the sensor ring resonator) or input from another sensor-such as a flow rate sensor, temperature sensor, etc. The analytical circuitry may include programming (e.g., stored instructions) for multiplexing or demultiplexing one or more signals (e.g., optical signals of a photodetector). The analytical circuitry may include one or more analog to digital converter (ADC), one or more digital to analog converter (DAC), one or more optoelectronic converter, one or more optical source (e.g., photodiode), and one or more detector (e.g., photodetector). The division between the photonic circuitry and the analytical circuitry may be arbitrary and descriptions herein should be understood to apply to elements which are described as located in the analytical circuitry instead of the photonic circuitry or in the photonic circuitry instead of the analytical circuitry. Analytical circuitry may include machine learning (ML) or artificial intelligence (AI) algorithms (e.g., stored or running on analytical circuitry), including algorithms which may improve thermal compensation (e.g., of detection algorithms).

In some embodiments, photonic circuitry may include one or more integrated light sources (e.g., optical sources). An integrated light source may be or contain one or more of a light emitting diode (LED), a coherent light source, a laser source, a broad-spectrum light source (e.g., broadband), etc. Photonic circuitry may include one or more filters, including one or more of a polarization filter, subwavelength grating, ring resonator filter, arrayed waveguide filter, etc. Photonic circuitry may include a first waveguide which optically couples a light source with the sensor ring resonator. Photonic circuitry may include one or more light sinks, absorber, or waveguide exit. Photonic circuitry may include a second waveguide which optically couples the sensor ring resonator with one or more ring resonators of an optical filter. Alternatively, or in addition, the sensor ring resonator may be cascade coupled with the one or more ring resonators (e.g., of the optical filter). The ring resonators of the optical filter may be of substantially the same geometry as the sensor ring resonator. In some embodiments, the resonators of the optical filter may be of substantially the same cross-section as the sensor ring resonator, but may have substantially different radii. The one or more ring resonators of the optical filter may be of a range of geometries (e.g., radii, cross-sectional areas, etc.) such that the one or more ring resonators are sensitive to (e.g., selectively filter) a range of wavelengths. The one or more ring resonators may have the same or different effective index of refraction.

When the sensor ring is exposed to the analyte, in some embodiments, it may experience a change in photonic characteristics, including a change in modal confinement, effective refractive index, etc. and thus experience a change in resonant wavelength. The sensor ring may act as a filter to select a first range of wavelengths from the wavelengths of the light within the first waveguide. The sensor ring may pass the first range of wavelengths to a second waveguide, which may be in optical communication with the one or more ring resonators of the optical filter. The one or more ring resonators of the optical filter may then be excited by the first range of wavelengths, where some of the one or more ring resonators may allow some of the first range of wavelengths to pass through while others or the one or more ring resonators may block at least some of the first range of wavelengths (e.g., filter). Each of the one or more ring resonators may be optically coupled to an output waveguide and may transmit a second range of wavelengths (e.g., filtered wavelengths) to the output waveguide. The wavelengths transmitted by each of the ring resonators (e.g., the filtered wavelengths) may correspond to binned or batched wavelength ranges, to multiple resonant modes, etc. The wavelengths which are transmitted (e.g., allowed) may then be detected, such as by determining which of the one or more ring resonators have optical throughput. The transmitted wavelengths may be detected by one or more detectors, including detectors corresponding to each of the output waveguides, a variable-wavelength detector, etc.

In some embodiments, the wavelength of the transmitted light may be determined based on which of the one or more ring resonators (e.g., filter resonators) it corresponds to. For an integrated chip, where the sensor ring resonator and the one or more ring resonators of the optical filter are fabricated substantially simultaneously, the fabrication geometry may function to reduce fabrication and thermal variation. Each of the ring resonators (e.g., the sensor ring resonator and the one or more ring resonators of the optical filter) may experience similar thermal conditions-if they are substantially co-located- and similar fabrication variations, when fabricated together, including as an integrated unit. The organization of the one or more ring resonators of the optical filter may compensate for thermal variations and fabrication variations.

In some embodiments, the fabrication process for the device may include fabrication of integrated passive photonics, fabrication of integrated active photonics, fabrication of microfluidic, fabrication of additional circuitry, which may include analytical circuitry, etc. In some embodiments, one or more components may be monolithically integrated. In some embodiments, one or more components may be heterogeneously integrated. The fabrication process may include joining of heterogeneous elements-through soldering, electrical bonding, through surface tension or adhesive (e.g., for joining of polymer microfluidic to silicon photonic devices), through the use of vias or metal connections, through the use of pins, etc.

Some embodiments may implement a ring resonator optically coupled to an optical filter comprises of ring resonators. Components of some embodiments may include the following:
Cascade connection of filter ring resonators to sensor ring resonator
Integrated photodetectors optically coupled to ring resonators of the optical filter
Integrated laser diode (or other light source) optically coupled to the sensor ring resonator
Subwavelength grating ring resonators as optical filter(s)
Subwavelength grating ring resonator as a sensor ring resonator
Slot ring resonators as optical filter(s)
Slot ring resonator as a sensor ring resonator
Self-compensating optical filter
Thermally compensated optical filter
Multiple stages of optical filters
Arrayed waveguide optical filter
Pre-filtering system optically coupled to the sensing ring resonator
Multiple sensing ring resonators and corresponding optical filters integrated into a single device
Multiple analyte sensitivity
Monolithically integrated passive photonics
Monolithically integrated active photonics
Monolithically integrated components, including electrical components
Heterogeneous integration, including of photonics and microfluidics
SiN, Si, Si-on-Insulator (SOI), Ge, SiGe, III-V, or polymer-based devices or components
Sensor ring functionalization

The structure of some embodiments may include the following:
Microfluidic channel fluidically coupled to the sensor ring resonator.
Microfluidic channel fluidically coupled to the ring resonators of the optical filter
Multiple sensing ring resonators optically, fluidically, electrically, etc. coupled
Multiplexed sensing devices
Analytical circuitry
A light source, including a laser source
Photodetectors

In operation, some embodiments may implement the following processes:
Fabrication of photonic devices
Fabrication of microfluidic devices
Operation of photonic devices
Operation of microfluidic devices
Detection of one or more analyte

Some embodiments are expected to afford the following advantages over other approaches, though it again should be emphasized that embodiments are not limited to systems that afford all of these advantages, which is not to suggest that any other description is limiting:
Lower cost
Thermal stability
Robustness to fabrication variation

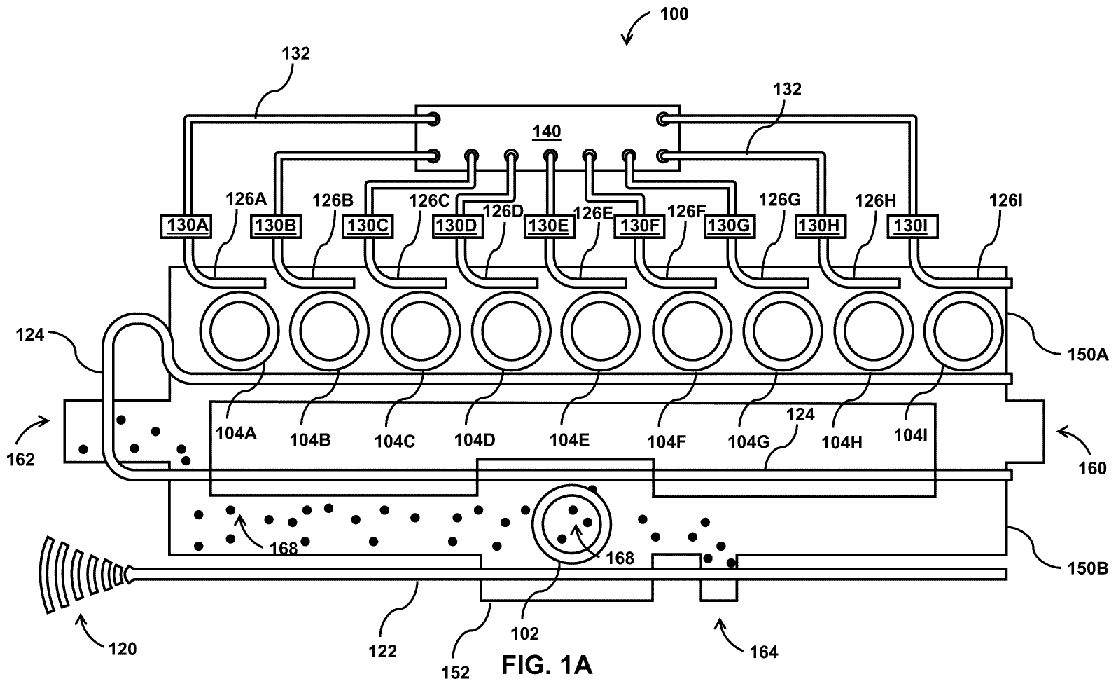

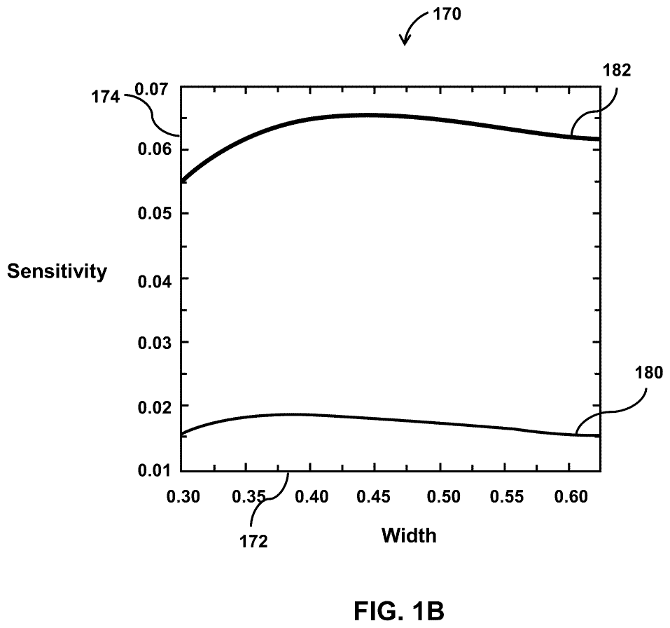

FIG. 1A is a schematic representation of a ring resonator based photonic circuit with split fluid channel for sensing. FIG. 1A depicts a sensing device 100 containing a sensor ring resonator 102 and multiple filter ring resonators 104A-104I. Nine filter ring resonators are depicted (e.g., the filter ring resonators 104A-104I), but the sensing device 100 may have more or fewer filter ring resonators. An optical source 120, here depicted as a coupler grating but which may be any appropriate optical source, is optically coupled to a first waveguide 122. The optical source 120 provides light, through optical coupling to the sensor ring resonator 102. The sensor ring resonator 102 is also optically coupled to a second waveguide 124. The second waveguide 124 is also optically coupled to the filter ring resonators 104A-104I. The sensor ring resonator 102 may act as a filter to select a range of wavelengths from the optical source 120 (via the first waveguide 122) and transmit that range of wavelengths to the second waveguide 124. Each of the filter ring resonators 104A-104I may act as a filter, selecting a range of wavelengths from the second waveguide 124. Each of the filter ring resonators 104A-104I may be tuned to a specific range of wavelengths. Each of the filter ring resonators 104A-104I may be optically coupled to an output waveguide, e.g., a corresponding one of waveguides 126A-1261. Each of the output waveguides may be coupled to a corresponding detector 130A-130I, which may be an optical detector, photodetector, etc. In some embodiments, each of the output waveguides may be coupled to a single detector (e.g., instead of detectors 130A-130I). The detectors 130A-130I may be communicatively coupled with a processing unit 140, which may be an ASIC chip, AI processing unit, etc., such as via communication lines 132.

The sensor ring resonator 102 may be located in a microfluidic channel 150B, which contains an analyte delivery fluid. The sensor ring resonator 102 may be located in a microfluidic chamber 152, which may be part of the microfluidic channel 150B. The filter ring resonators 130A-130I may be located in a microfluidic channel 150A, which also contains the analyte deliver fluid. The analyte delivery fluid may be introduced, such as into both the microfluidic channel 150A and the microfluidic channel 150B, through a fluid entry port 160. The analyte delivery fluid may exit the microfluidic channel 150A and the microfluidic channel 150B through a fluid exit port 162. The microfluidic channel may be arranged in any appropriate manner. For example, the microfluidic channel 150A and the microfluidic channel 150B may have separate fluid entry and exit ports and may or may not be fluidically coupled. An analyte 168 may be introduced into the analyte delivery fluid through an analyte entry port 164. The analyte 168 may be a virus or any appropriate analyte, including biological material.

In some embodiments, the proposed sensing architecture (e.g., of FIG. 1A) may offer a spectral accuracy <5 picometers (pm). This may facilitate the detection of ultralow virus loads without requiring expensive spectral measurements. Furthermore, the complementary metal-oxide-semiconductor (CMOS) compatibility of the components used in the sensing architecture may facilitate high volume manufacturing, and thus lower the cost. In some embodiments, the sensing architecture may combine the flexibility of electronics and the sensitivity of photonics to enable sensing and signal processing (e.g., on-sensor AI) on a single platform using low-cost, low-complexity optoelectronic circuits.

FIG. 1A shows the schematic top view of the sensor device 100 which consists of the sensor ring resonator 102 cascaded with multiple filter ring resonators 104A-104I via the drop port of the sensor ring. Each of the filters (e.g., the filter ring resonators 104A-104I) is designed with a specific ring radius such that these rings are resonant to the frequencies separated by 25 GHz. The resonant wavelength represents the center wavelength of the respective channel of the on-chip spectrometer. The drop port of each filter ring resonator 104A-104I is coupled to a separate photodetector (e.g., detector 130A-1031) to measure power received from each channel. The photodetector array is connected to the processing unit 140 (e.g., an AI unit) to process the electrical signal to obtain the quantitative measurement of the viral/pathogen load.

To achieve fabrication tolerant and temperature insensitive sensor response, the cross-sectional waveguide geometry of the sensor and filter rings are designed to be identical. Moreover, the microfluidic channels (e.g., the microfluidic channel 150A and the microfluidic channel 150B) covers the sensor ring resonator 102 as well as filter ring resonators 104A-104I to ensure uniform top cladding for the sensor and filter rings. Consequently, any global variations (temperature or structural variation) may be self-compensated.

FIG. 1B is graph 170 depicting example waveguide sensitivity to transverse electric (TE) and transverse magnetic (TM) modes. The graph 170 depicts waveguide sensitivity, given by a change in effective index Δη_{eff} divided by a change in cladding index Δη_{c} on a y-axis 174 as a function of waveguide width (W) in μm on an x-axis 172 for a wavelength of λ=1550 nm. The TE mode corresponds to line 180, while the TM mode corresponds to line 182. In the example, waveguide height is assumed to be 220 nm. A change in effective index (Δη_{eff}) with respect to the change in cladding index (Δn_{c}) is used to determine waveguide sensitivity. FIG. 1B clearly shows that the sensitivity for TM polarization is nearly 3 times higher than TE polarization resulting from the lower confinement of TM polarized light due to the lower aspect ratio (H/W) of the waveguide geometry. In some embodiments, the sensor may be designed for TM polarization to take advantage of the increased TM waveguide sensitivity.

3D FDTD simulation of the entire sensor circuit may be prohibitively complex, but the sensor device has been modeled using Lumerical Interconnect by obtaining bend-induced loss and coupling coefficients from a 3D FDTD model and η_{eff} from Lumerical mode solutions. As the mode is weakly confined for the TM polarization, it may be important to optimize the bend radius for the sensor ring resonator and filter ring resonators. Based on a 3D FDTD simulation for 90-degree bends, there may be an estimated a loss of 0.04 dB and 0.013 dB for bends of radius 10 μm and 15 μm, respectively, which may be the radii of the sensor ring resonator and the filter ring resonators, respectively. The bend induced loss in the interconnect simulations may be modeled as propagation loss and added to nominal propagation loss (e.g., of 2 dB/cm) to give a modeled propagation loss of 27.5 dB/cm for the sensor ring resonator (with a radius of 10 μm) and 7.5 dB/cm for the filter ring resonators (with radii of 15 μm). Bus to ring and ring to drop coupling may be designed to have a 5% to have a maximum quality factor.

In some embodiments, the functionalized layer and the corona virus may be modeled as a 15 nm thick functionalized layer and a solid sphere of diameter 100 nm with a refractive index of 1.45 and 1.5, respectively. In some embodiments, in a simulation, a part of the sensor ring resonator is modeled as (e.g., replaced by) a waveguide with a modified effective index reflective of a virus on top of the waveguide, with a length of N×100 nm, where N is the number of viruses modeled as attached on the sensor surface. For simplicity, each virus may be considered as attached on the middle of the waveguide top surface.

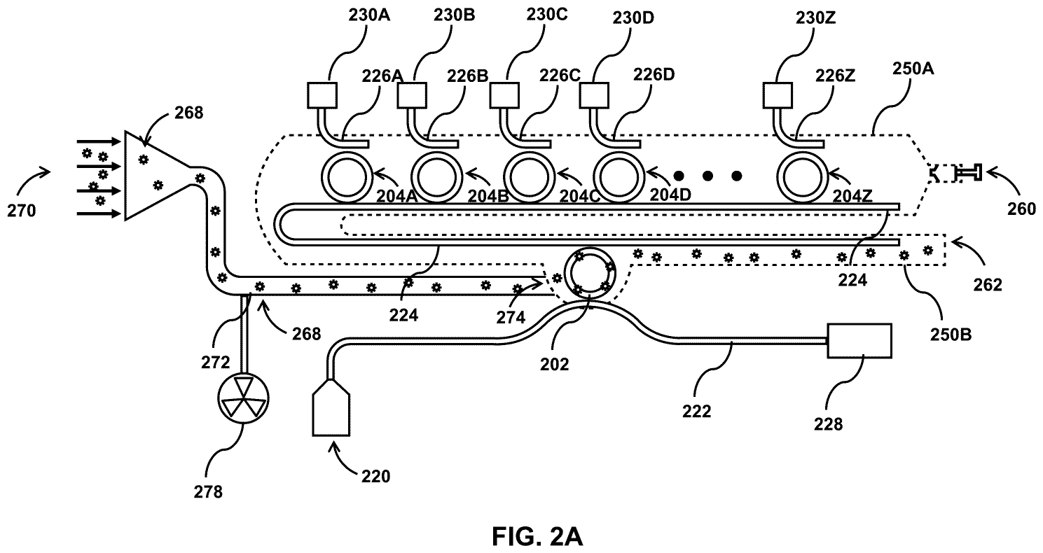

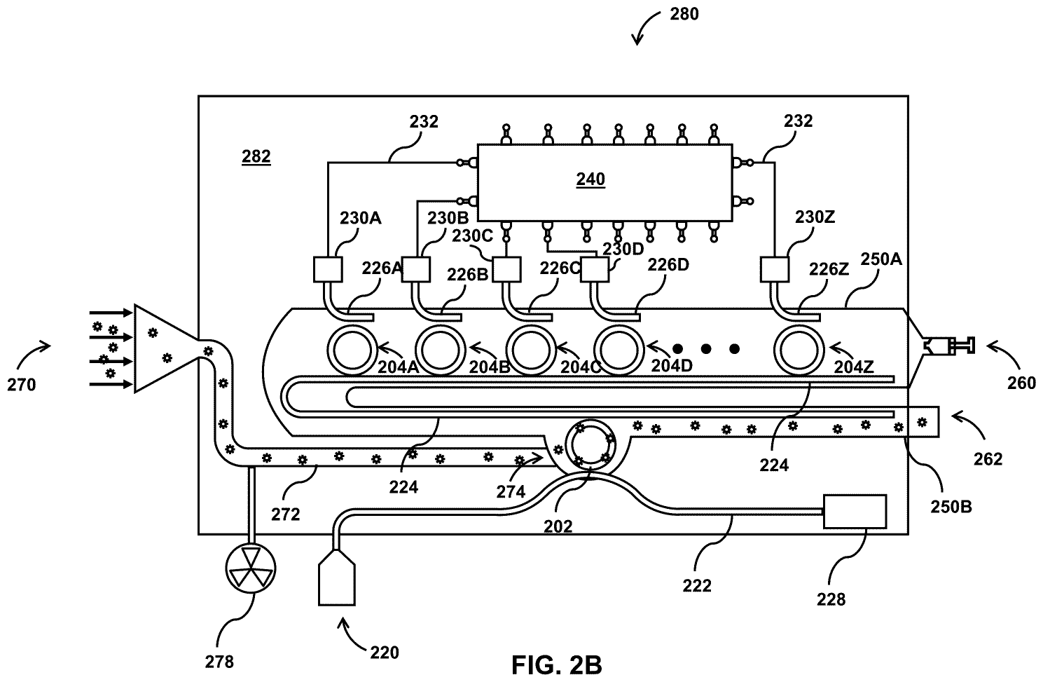

FIG. 2A is a schematic representation of a ring resonator based photonic circuit with a single fluid channel for sensing. FIG. 2A depicts a sensing device containing a sensor ring resonator 202 and multiple filter ring resonators 204A-204Z. Five filter ring resonators are depicted (e.g., the filter ring resonators 204A-204D and 204Z), but the sensing device may have more or fewer filter ring resonators. An optical source 220, which may be any appropriate optical source, is optically coupled to a first waveguide 222. The first waveguide 222 terminates in an absorber 228 or other device which may operate to prevent reflectance of an optical signal from a waveguide termination.

The optical source 220 provides light, through optical coupling to the sensor ring resonator 202. The sensor ring resonator 202 is also optically coupled to a second waveguide 224. The second waveguide 224 is also optically coupled to the filter ring resonators 204A-204Z. The sensor ring resonator 202 may act as a filter, such as previously described in reference to the sensor ring resonator 102 of FIG. 1A, to select a range of wavelengths from the optical source 220 (via the first waveguide 222) and transmit that range of wavelengths to the second waveguide 224. Each of the filter ring resonators 204A-204Z may act as a filter, selecting a range of wavelengths from the second waveguide 224, such as previously described for the filter ring resonators 104A-104I of FIG. 1A. Each of the filter ring resonators 204A-204Z may be tuned to a specific range of wavelengths. Each of the filter ring resonators 204A-204Z may be optically coupled to an output waveguide, e.g., a corresponding one of waveguides 226A-226Z. Each of the output waveguides may be coupled to a corresponding detector 230A-230Z, which may be an optical detector, photodetector, etc. The detectors 230A-230Z may be communicatively coupled with a processing unit (not depicted), which may be an ASIC chip, AI processing unit, etc.

The sensor ring resonator 202 may be located in a microfluidic channel 250B, which contains an analyte delivery fluid. The filter ring resonators 204A-204Z may be located in a microfluidic channel 250A, which also contains the analyte deliver fluid. The analyte delivery fluid may be introduced, such as into the microfluidic channel 150A, through a fluid entry port 260. The analyte delivery fluid may flow through the microfluidic channel 250A into the microfluidic channel 250B. The analyte delivery fluid may exit the microfluidic channel 150B through a fluid exit port 262. An analyte 268 may be introduced into the analyte delivery fluid through an analyte entry port 270. The analyte 268 may be a virus or any appropriate analyte, including biological material. The analyte 268 may be propelled into the analyte delivery fluid, such as by a pump 278, along a microfluidic channel 272. The analyte 268 may enter the analyte delivery fluid at an interface 274. The interface 274 may be a fluid interface, such as an air-liquid interface.

FIG. 2B is a schematic representation of a ring resonator based photonic circuit for sensing integrated in a fluid analyzer 280. FIG. 2B depicts the sensing device of FIG. 2A integrated into a subtracted. The detectors 230A-230Z may be communicatively coupled, such as via communication lines 232, with a processing unit 240, which may be an ASIC chip, AI processing unit, etc. The sensor ring resonator 202 and the filter ring resonators 204A-204Z may be integrated into a substrate 282, which may be a SOI substrate. In some embodiments, the microfluidic channels (e.g., the microfluidic channel 250A and the microfluidic channel 250B) may also be integrated into the substrate 282. In some embodiments, the processing unit 240, which may instead or additionally be a memory unit, may be integrated into the substrate 282, such as homogeneously through CMOS integration, heterogeneously through bump soldering, etc.

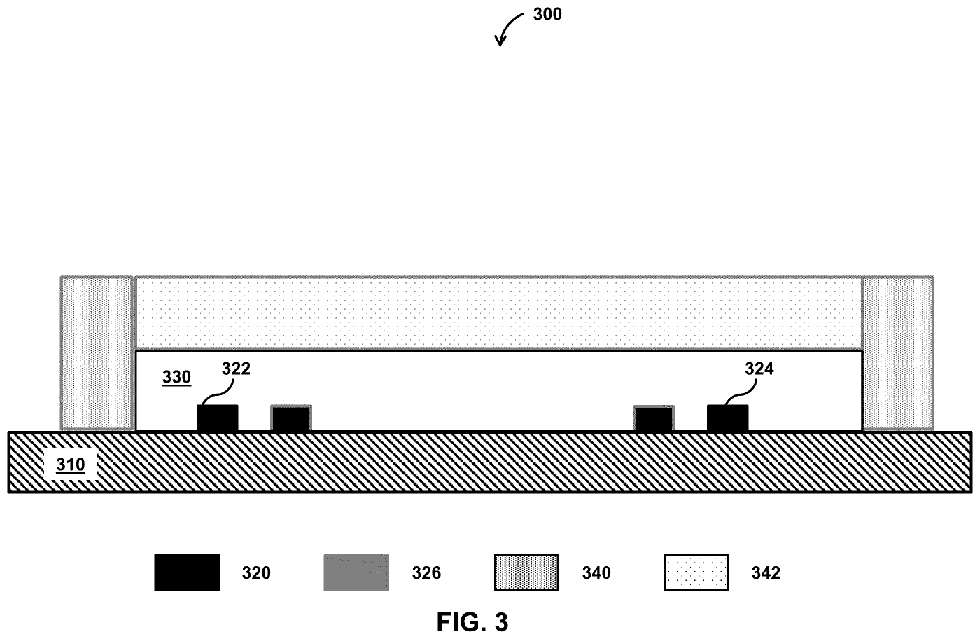

FIG. 3 is a schematic representation of a cross-section 300 of a ring resonator based photonic circuit. The ring resonator may be fabricated of silicon 320 or any other appropriate semiconducting material. The ring resonator may be fabricated on a substrate 310, which may be a silicon-on-insulator (SOI) substrate. The ring resonator may be fabricated for the same material (e.g., silicon 320) as one or more waveguides (e.g., an input waveguide 322 and an output waveguide 324). The waveguides may be optical fibers. The waveguides may be any appropriate waveguide geometry. The waveguides and the ring resonator may have the same or different cross-sectional area (e.g., width (W) and height (H)). The waveguides and ring resonator may have any appropriate separation, such as a separation governed by the optical interference length of a signal operating on the waveguide. The ring resonator may have any appropriate radius, including a variable radius. For example, the filter ring resonators may have different radii, which may allow them to operate as a filter. The ring resonator may be coated with a functionalization layer 326. The functionalization layer may coat the ring resonator and the waveguides, or the ring resonator(s) only. The functionalization layer may be a top coating or may coat multiple sides of the ring resonator.

The ring resonator may lie in a microfluidic channel surrounded by sidewalls 340 and ceiling 342. The microfluidic channel may instead be open to atmosphere (e.g., not surrounded by a ceiling 342). The microfluidic channel may contain an analyte delivery fluid 330, which may be any appropriate fluid including a gas, atmosphere, a liquid, etc.

The ring resonator may be fabricated on the substrate 310 by any appropriate method, including photolithography, directional etching, shadowmasking, etc. The ring resonator may be fabricated by subtractive fabrication, by additive fabrication, etc. The fabrication of the ring resonator may include fabrication of passive silicon devices, such as waveguides, resistors, gratings, etc. The fabrication of the ring resonator may include fabrication of active silicon devices, such as memory circuits (e.g., storage), processor circuits, etc. Devices referred to as active and passive silicon devices may instead be active and passive devices of any other appropriate material, such as GaAs, SiN, etc.

The microfluidic channel may be fabricated on the substrate 310 by any appropriate method, including photolithography, directional etching, shadowmasking, etc. The microfluidic channel may be heterogeneously integrated onto the ring resonator and substrate. The microfluidic channel may be fabricated, such as in a polymer, by stamp imprinting, molding, etc. The microfluidic channel may be fabricated from a semiconducting material, such as from silicon.

Processing and memory circuitry may be integrated into the ring resonator or substrate. Various processing and memory devices may be fabricated into the substrate 310, including before or after the fabrication of the ring resonator. Processing and memory circuitry may be joined to the substrate, such as via a through pin scheme, soldering, etc. The ring resonators and waveguides may be optically and electrically coupled (e.g., communicatively coupled) with processing and memory circuitry.

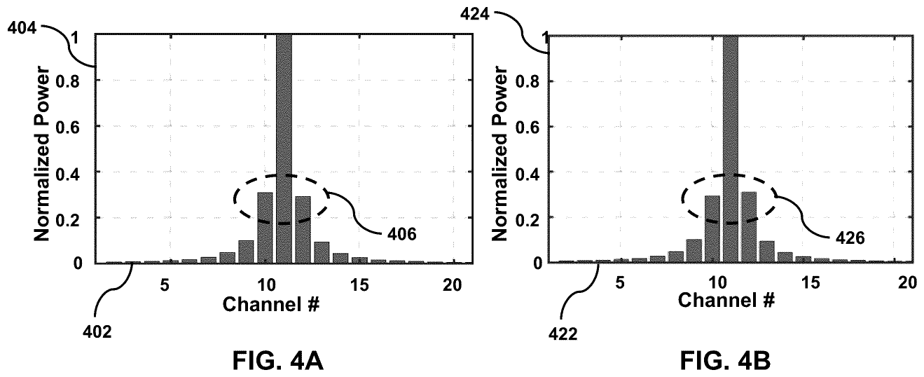

FIGS. 4A-4B are graphs depicting a response of a ring resonator based photonic circuit to exposure to a virus particle. FIG. 4A shows an example on-chip spectrometer response without virus, while FIG. 4B shows an example on-chip spectrometer response with a virus. FIG. 4A depicts channel response in normalized power arbitrary units along a y-axis 404 as a function of channel along an x-axis 402. The x-axis 402 shows channels, each representing a specific filter ring resonator. FIG. 4B depicts channel response in normalized power arbitrary units along a y-axis 424 as a function of channel along an x-axis 422. The x-axis 422 shows channels corresponding to those of the x-axis 402 of FIG. 4A, each representing a specific filter ring resonator.

In the example, FIG. 4A shows that, in the absence of virus (e.g., in FIG. 4A), power at channel #10 is higher than that of channel #12 (as identified by dotted ellipse 406). Whereas when one virus is present on the sensor surface (e.g., in FIG. 4B), channel #12 receives higher power compared to channel #10 (as identified by dotted ellipse 426) indicating that the sensor spectrum is red shifted by presence of the virus particle. The spectral shift by using equation 1, below:

$${λ}_{shift} = \frac{{\sum }_{i = 1}^{n} {P}_{i} \times {λ}_{i}}{{\sum }_{i = 1}^{n} {P}_{i}} \qquad ( 1 )$$

where Pᵢ and λᵢ are the optical power and center wavelength at the ith channel. For a single virus, the wavelength shift may be estimated to be 6.4 pm from the photodetector (10 nW sensitivity) response proving that the device is capable of detecting even a single virus. The estimated wavelength shift may be ˜1.4 pm offset from the actual spectral shift of 5 pm. This gives an accuracy of <5 pm, which may not be possible with conventional intensity/power-based sensing techniques.

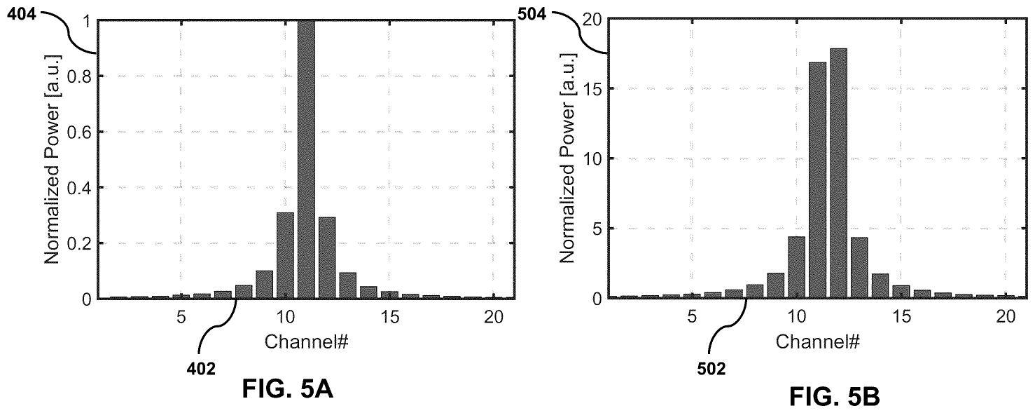

FIGS. 5A-5B are graphs depicting a response of a ring resonator based photonic circuit to exposure to multiple virus particles. FIG. 5A is the graph of FIG. 4A, reproduces for ease of description. FIG. 5A shows an example on-chip spectrometer response without virus, while FIG. 5B shows an example on-chip spectrometer response with a 10 viruses. FIG. 5B depicts channel response in normalized power arbitrary units along a y-axis 504 as a function of channel along an x-axis 502. The x-axis 502 shows channels corresponding to those of the x-axis 402 of FIG. 4A, each representing a specific filter ring resonator.

In the example, FIG. 5A shows that, in the absence of virus (e.g., in FIG. 5A), power at channel #10 is higher than that of channel #12, while channel #11 shows a local maximum in response. Whereas when 10 viruses are present on the sensor surface (e.g., in FIG. 5B), channel #12 receives higher power (e.g., a local maximum) compared to channel #10 and channel #11 indicating that the sensor spectrum is red shifted by presence of the virus particle.

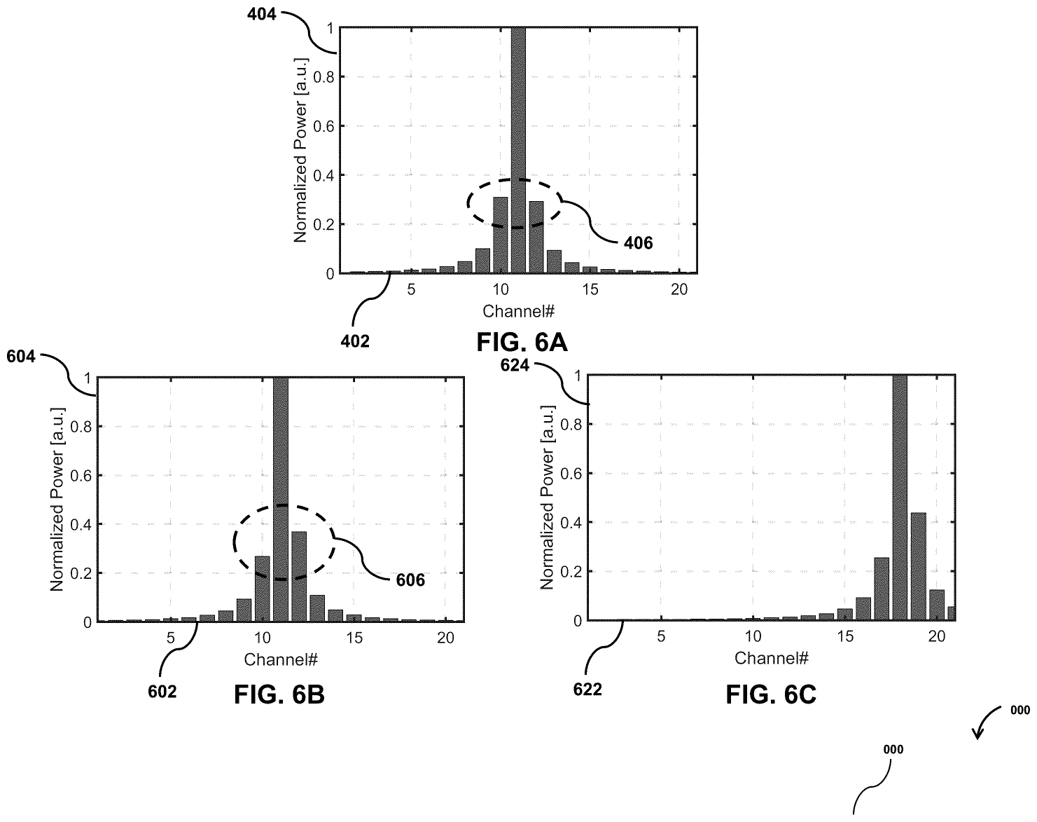

FIGS. 6A-6C are graphs depicting responses of a ring resonator based photonic circuit to different virus exposure levels. FIG. 6A is the graph of FIG. 4A, reproduces for case of description. FIG. 6A shows an example on-chip spectrometer response without virus, while FIG. 6B shows an example on-chip spectrometer response with 5 viruses and FIG. 6C shows an example on-chip spectrometer response with 275 viruses. FIG. 6B depicts channel response in normalized power arbitrary units along a y-axis 604 as a function of channel along an x-axis 602. The x-axis 602 shows channels corresponding to those of the x-axis 402 of FIG. 6A, each representing a specific filter ring resonator. FIG. 6C depicts channel response in normalized power arbitrary units along a y-axis 624 as a function of channel along an x-axis 622. The x-axis 622 shows channels corresponding to those of the x-axis 402 of FIG. 6A, each representing a specific filter ring resonator.

In the example, FIG. 6A shows that, in the absence of virus (e.g., in FIG. 6A), power at channel #10 is higher than that of channel #12 (as indicated by the dotted ellipse 406), while channel #11 shows a local maximum in response. Whereas when 5 viruses are present on the sensor surface (e.g., in FIG. 6B), channel #12 receives higher power compared to channel #10 (as indicated by a dotted ellipse 606) that the sensor spectrum is red shifted by presence of the virus particle. The relative strength of the side channels (e.g., channels #10 and #12) has shifted, but for both 0 and 5 viruses, the local maximum remains at channel #11.

Whereas when 275 viruses are present on the sensor surface (e.g., in FIG. 6C), channel #12 receives higher power compared to channel #10, but the local maximum in power has been shifted to channel #18, with primary side channels #17 and #18. This may indicated that the sensor spectrum is red shifted by presence of the virus particle, but that the red shift is not linear with number of virus particle (which agrees with Equation 1).

Temperature and fabrication conditions may also affect resonant wavelengths of ring resonators. However, architecture may be used to compensate for shifts due to thermal and fabrication variations.

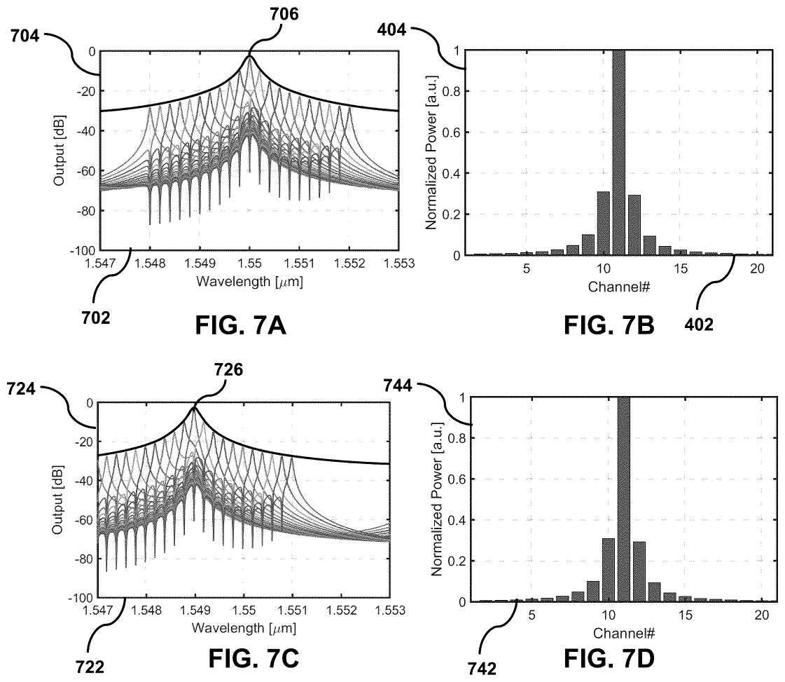

FIGS. 7A-7D are graphs depicting responses of a ring resonator based photonic circuit for different fabrication conditions. FIGS. 7A and 7C depict absolute spectral response of sensor and filter rings for a first geometry at room temperature and a second geometry and an elevated temperature, respectively. FIGS. 7B and 7D depict on-chip spectral responses for the first geometry at room temperature and the second geometry at the elevated temperature, respectively. FIG. 7B is the graph of FIG. 4A, reproduces for case of description.

In order to estimate the temperature dependence and fabrication tolerance of the circuit, simulation results for a first structure, which may be an ideal or ideally-fabricated structure, at room temperature (300 K) and second structure, which may be a non-ideal or inaccurately-fabricated structure, at elevated temperature (310 K). FIG. 7A depicts spectral output of the sensor ring resonator as line 706, and various spectral outputs of the filter ring resonators as a function of output (in dB) along y-axis 704 versus wavelength (in μm) along x-axis 702. FIG. 7C depicts output of the sensor ring resonator as line 726, and various spectral outputs of the filter ring resonators as a function of output (in dB) y-axis 724 versus wavelength (in μm) along x-axis 722. As can be seen in the difference of FIG. 7A and FIG. 7B, the sensor spectrum is blue shifted (e.g., the peak of the line 706 lies at about 1.55 μm while the peak of the line 726 lies at about 1.549 μm), showing that structural dependency may be more significant than temperature dependence. That is, structural dependency may blue shift ring resonator response, while temperature dependence may red shift response. In such as case, the relative strength of the respective shifts may determine the overall shift of the response due to thermal and fabrication variation.

FIG. 7B depicts the example on-chip spectrometer response without virus, as depicted in FIG. 4A. FIG. 7B depicts channel response in normalized power arbitrary units along a y-axis 404 as a function of channel along an x-axis 402, for the device of FIG. 7A. The x-axis 402 shows channels, each representing a specific filter ring resonator. FIG. 7D depicts channel response in normalized power arbitrary units along a y-axis 744 as a function of channel along an x-axis 742 for the device of FIG. 7B. The x-axis 742 shows channels, each representing a specific filter ring resonator and corresponding to the channels of FIG. 7B. FIG. 7B depicts a response for first geometry and temperature (W=500 nm, H=220 nm, T=300 K), while FIG. 7D depicts a response for a second (non-ideal) geometry and elevated temperature (W=495 nm, H=220 nm, T=310 K). While geometry, temperature, and spectral response are different for the devices of FIGS. 7B and 7C, the on-chip spectrometer spectrum remains the same, independent of the geometry and temperature change. This may be because the spectral shift for the sensor ring resonators and filter ring resonators are the same for the global variations. Consequently, the respective photodetector receives the same optical power independent of temperature and geometry variations.

A major challenge associated with quantitative sensing is the location of attachment of the virus. As the device is designed to operate at TM polarization, the device may be more sensitive when the virus attaches on the top surface. It may be less sensitive when the virus is attached to the sidewalls. This sensitivity difference may be mediated by functionalizing the top-surface of the waveguide alone. Additionally, the maximum detectable viral load may be limited by the free spectral range (FSR) of the senor ring resonator and filter ring resonators. However, the device may be capable of sensing ultra-low pathogen concentrations as small as a single virus with a fabrication tolerant and temperature insensitive architecture.

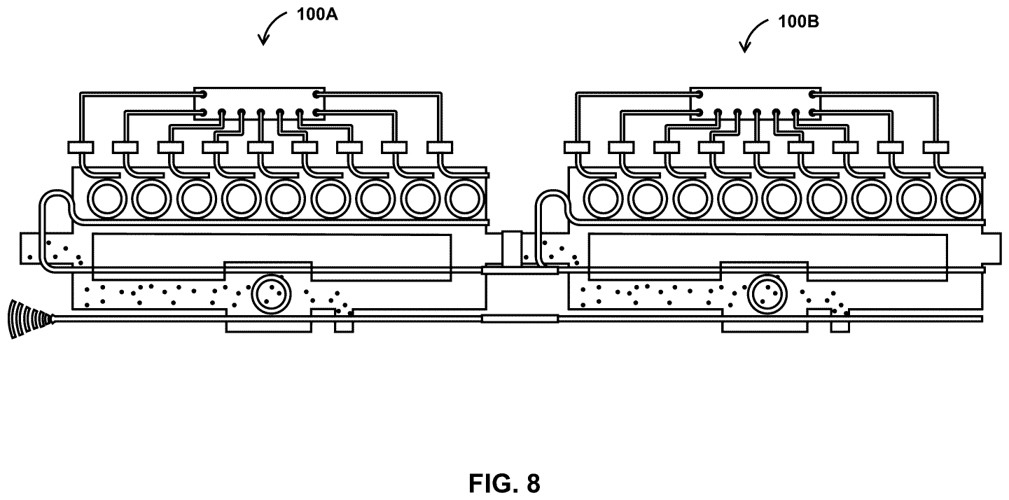

FIG. 8 is a schematic representation of a series of ring resonator based photonic circuits. FIG. 8 depicts two sensing devices, e.g., sensing device 100A and sensing device 100B, in series. FIG. 8 shows multiple sensing devices, which are depicted as the sensing device 100 of FIG. 1A, but which may be any appropriate sensing device, such as the sensing devices of FIG. 2A or FIG. 2B. FIG. 8 depicts two sensing devices in series, but multiple sensing devise (e.g., three or more) may be connected instead or additionally. FIG. 8 depicts sensing devices in series, but sensing devices may instead or additionally be connected in parallel.

In some embodiments, when sensing devices are connected, a sensor ring resonator may be optically coupled, such as through a cascade coupling, with multiple filter ring resonators. In some embodiments, multiple senor ring resonators may be optically coupled to multiple filter ring resonators, including each to a set of multiple ring resonators. In some embodiments, multiple sensor ring resonators may occupy a microfluidic channel. In some embodiments, multiple sensor ring resonators may be optically coupled to an input waveguide (e.g., first waveguide 122). In some embodiments, multiple sensor ring resonators may have substantially similar geometries and resonant wavelengths. In some embodiments, multiple sensor ring resonators may have substantially different resonant wavelengths.

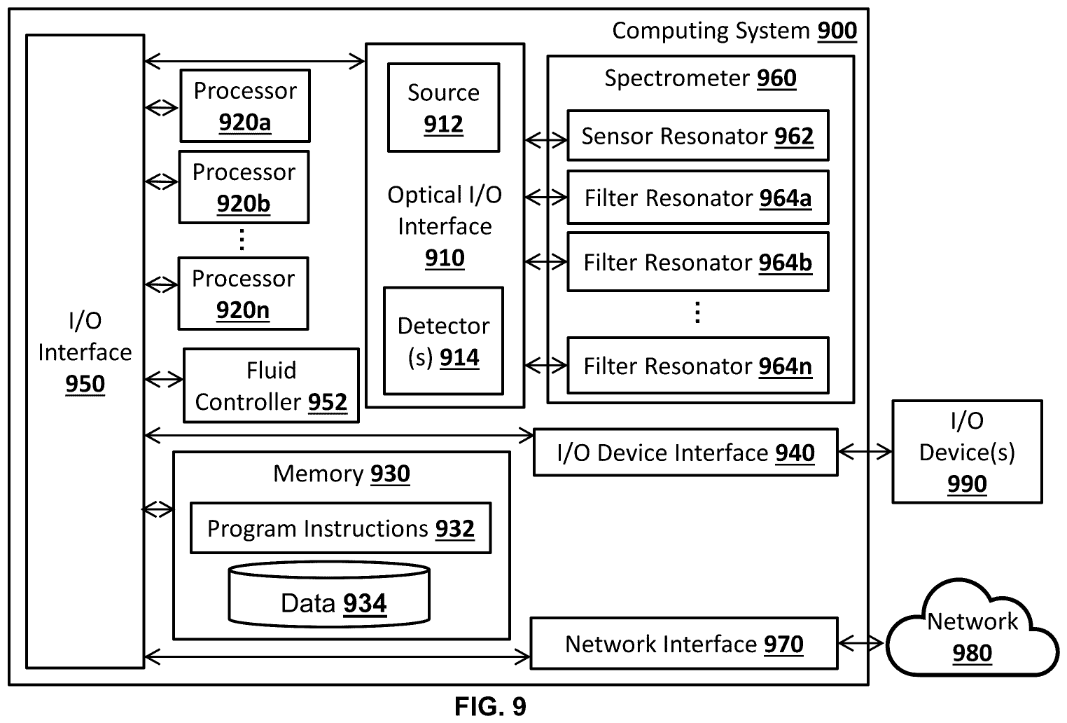

FIG. 9 illustrates an example computing system with a ring resonator photonic circuit. Various portions of systems and methods described herein may include or be executed on one or more computing systems similar to computing system 900. Further, processes and modules described herein may be executed by one or more processing systems similar to that of computing system 600.

Computing system 900 may include one or more processors (e.g., processors 920a-920n) coupled to system memory 930, a I/O interface 940, and a network interface 970 via an input/output (I/O) interface 950. A processor may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computing system 900. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 930). Computing system 900 may be a uni-processor system including one processor (e.g., processor 920a-920n), or a multi-processor system including any number of suitable processors (e.g., 920a-920n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus may also be implemented as, special purpose logic circuitry, e.g., an FPGA (field programmable gate array) or an ASIC (application specific integrated circuit). Computing system 900 may include a plurality of computing devices (e.g., distributed computing systems) to implement various processing functions.

Computing system 900 may include a spectrometer 960, which may contain one or more sensor resonator 962 and one or more filter resonator (e.g., filter resonators 964a-964n), coupled to an analog optical I/O interface 910. The optical I/O interface 910 may be coupled to the I/O interface 950. The spectrometer 960 may be coupled to the memory 930 or the one or more processors 920a-920n through the optical I/O interface 910 and the I/O interface 950. The spectrometer 960 may instead be coupled, such as through the processors 920a-920n, to a system memory 930, and a user interface 940 via an input/output (I/O) interface 950. The sensor resonator 962 and the one or more filter resonators 964a-964n may be interrogated by one or more optical element, a source 912 and one or more detectors 914. The source 912 may be part of the optical I/O interface 910 or in communication with the optical I/O interface 910. The detector(s) may be part of the optical I/O interface 910 or in communication with the optical I/O interface 910. The source 912 and the detector(s) 914 may be in communication with the I/O interface 950 or controlled by one or more of the processors 920a-920n. The sensor resonator 962 and the one or more filter resonators 964a-964n may be exposed to input, such as an analyte or other material to which they are sensitive. The sensor resonator 962 and the one or more filter resonators 964a-964n may comprise ring resonators or any other appropriate resonators.

The I/O interface may be coupled to a fluid controller 952. The fluid controller 952 may contain memory, processors, motors, etc. The fluid controller 952 may operate to deliver fluid, including analyte delivery fluid, to the sensor resonator 962 and the one or more filter resonators 964a-964n. The fluid controller 952 may operate a pump. The fluid controller 952 may be controlled by the processors 920a-920n through the I/O interface 950.

The I/O device interface 940 may comprise one or more I/O device interface, for example to provide an interface for connection of one or more I/O devices 990 to computing system 900. The I/O device interface 940 may include devices that receive input (e.g., from a user) or output information (e.g., to a user). The I/O device interface 940 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. The I/O device interface 940 may be connected to computing system 900 through a wired or wireless connection. The user interface 940 may be connected to computing system 900 from a remote location. The user interface 940 may be in communication with one or more other computing systems. Other computing units, such as located on remote computer system, for example, may be connected to computing system 900 via a network 980, which may be connected via the network interface 970.

System memory 930 may be configured to store program instructions 932 or data 934. Program instructions 932 may be executable by a processor (e.g., one or more of processors 920a-920n) to implement one or more embodiments of the present techniques. Program instructions 932 may include modules of computer program instructions for implementing one or more techniques described herein with regard to various processing modules. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network, such as the network 980.

System memory 930 may include a tangible program carrier having program instructions stored thereon. A tangible program carrier may include a non-transitory computer readable storage medium. A non-transitory computer readable storage medium may include a machine-readable storage device, a machine-readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random-access memory (SRAM), synchronous dynamic RAM (SDRAM)), bulk storage memory (e.g., CD-ROM and/or DVD-ROM, hard-drives), or the like. System memory 930 may include a non-transitory computer readable storage medium that may have program instructions stored thereon that are executable by a computer processor (e.g., one or more of processors 920a-920n) to cause the subject matter and the functional operations described herein. A memory (e.g., system memory 930) may include a single memory device and/or a plurality of memory devices (e.g., distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times.

I/O interface 950 may be configured to coordinate I/O traffic between processors 920a-920n, optical I/O interface 910, spectrometer 960, source 912, detector(s) 914, sensor resonator 962, one or more filter resonators 964a-964n, system memory 930, I/O device interface 940, etc. I/O interface 950 may perform protocol, timing, or other data transformations to convert data signals from one component (e.g., system memory 930) into a format suitable for use by another component (e.g., processors 920a-920n). I/O interface 950 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computing system 900 or multiple computing systems 900 configured to host different portions or instances of embodiments. Multiple computing systems 900 may provide for parallel or sequential processing/execution of one or more portions of the techniques described herein.

Those skilled in the art will appreciate that computing system 900 is merely illustrative and is not intended to limit the scope of the techniques described herein. Computing system 900 may include any combination of devices or software that may perform or otherwise provide for the performance of the techniques described herein. For example, computing system 900 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, or a Global Positioning System (GPS), or the like. Computing system 900 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computing system 900 may be transmitted to computing system 900 via transmission media or signals such as electrical, electromagnetic, or digital signals, conveyed via a communication medium such as a network (e.g., the network 980) or a wireless link. Various embodiments may further include receiving, sending, or storing instructions or data implemented in accordance with the foregoing description upon a computer-accessible medium. Accordingly, the present techniques may be practiced with other computer system configurations.

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted, for example such software or hardware may be intermingled, conjoined, replicated, broken up, distributed (e.g., within a data center or geographically), or otherwise differently organized. The functionality described herein may be provided by one or more processors of one or more computers executing code stored on a tangible, non-transitory, machine-readable medium. In some cases, notwithstanding use of the singular term “medium,” the instructions may be distributed on different storage devices associated with different computing devices, for instance, with each computing device having a different subset of the instructions, an implementation consistent with usage of the singular term “medium” herein. In some cases, third party content delivery networks may host some or all of the information conveyed over networks, in which case, to the extent information (e.g., content) is said to be supplied or otherwise provided, the information may be provided by sending instructions to retrieve that information from a content delivery network.

The reader should appreciate that the present application describes several independently useful techniques. Rather than separating those techniques into multiple isolated patent applications, applicants have grouped these techniques into a single document because their related subject matter lends itself to economies in the application process. But the distinct advantages and aspects of such techniques should not be conflated. In some cases, embodiments address all of the deficiencies noted herein, but it should be understood that the techniques are independently useful, and some embodiments address only a subset of such problems or offer other, unmentioned benefits that will be apparent to those of skill in the art reviewing the present disclosure. Due to costs constraints, some techniques disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such techniques or all aspects of such techniques.

It should be understood that the description and the drawings are not intended to limit the present techniques to the particular form disclosed, but to the contrary, the intention is to cover all modifications, equivalents, and alternatives falling within the spirit and scope of the present techniques as defined by the appended claims. Further modifications and alternative embodiments of various aspects of the techniques will be apparent to those skilled in the art in view of this description. Accordingly, this description and the drawings are to be construed as illustrative only and are for the purpose of teaching those skilled in the art the general manner of carrying out the present techniques. It is to be understood that the forms of the present techniques shown and described herein are to be taken as examples of embodiments. Elements and materials may be substituted for those illustrated and described herein, parts and processes may be reversed or omitted, and certain features of the present techniques may be utilized independently, all as would be apparent to one skilled in the art after having the benefit of this description of the present techniques. Changes may be made in the elements described herein without departing from the spirit and scope of the present techniques as described in the following claims. Headings used herein are for organizational purposes only and are not meant to be used to limit the scope of the description.

As used throughout this application, the word “may” is used in a permissive sense (e.g., meaning having the potential to), rather than the mandatory sense (e.g., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, e.g., encompassing both “and” and “or.” The term “each” does not require an exact relationship or that absolutely all elements thus described are involved, e.g., each may indicate substantially all and does not require participation of all elements identified as each. The term “each” may indicate a substantially one-to-one relationship, a one-to-many relationship, etc. Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Similarly, reference to “a computer system” performing step A and “the computer system” performing step B may include the same computing device within the computer system performing both steps or different computing devices within the computer system performing steps A and B. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, e.g., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X′ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device. Features described with reference to geometric constructs, like “parallel,” “perpendicular/orthogonal,” “square”, “cylindrical,” and the like, should be construed as encompassing items that substantially embody the properties of the geometric construct, e.g., reference to “parallel” surfaces encompasses substantially parallel surfaces. The permitted range of deviation from Platonic ideals of these geometric constructs is to be determined with reference to ranges in the specification, and where such ranges are not stated, with reference to industry norms in the field of use, and where such ranges are not defined, with reference to industry norms in the field of manufacturing of the designated feature, and where such ranges are not defined, features substantially embodying a geometric construct should be construed to include those features within 15% of the defining attributes of that geometric construct. The terms “first”, “second”, “third,” “given” and so on, if used in the claims, are used to distinguish or otherwise identify, and not to show a sequential or numerical limitation. As is the case in ordinary usage in the field, data structures and formats described with reference to uses salient to a human need not be presented in a human-intelligible format to constitute the described data structure or format, e.g., text need not be rendered or even encoded in Unicode or ASCII to constitute text; images, maps, and data-visualizations need not be displayed or decoded to constitute images, maps, and data-visualizations, respectively; speech, music, and other audio need not be emitted through a speaker or decoded to constitute speech, music, or other audio, respectively. Computer implemented instructions, commands, and the like are not limited to executable code and may be implemented in the form of data that causes functionality to be invoked, e.g., in the form of arguments of a function or API call. To the extent bespoke noun phrases (and other coined terms) are used in the claims and lack a self-evident construction, the definition of such phrases may be recited in the claim itself, in which case, the use of such bespoke noun phrases should not be taken as invitation to impart additional limitations by looking to the specification or extrinsic evidence.

In this patent, to the extent any U.S. patents, U.S. patent applications, or other materials (e.g., articles) have been incorporated by reference, the text of such materials is only incorporated by reference to the extent that no conflict exists between such material and the statements and drawings set forth herein. In the event of such conflict, the text of the present document governs, and terms in this document should not be given a narrower reading in virtue of the way in which those terms are used in other materials incorporated by reference.

The present techniques will be better understood with reference to the following enumerated embodiments:

1. A device for analyte detection, comprising: a first waveguide; a sensing ring resonator, optically coupled to the first waveguide, wherein the sensing ring resonator is sensitive to an analyte; multiple ring resonators optically coupled to the sensing ring resonator, one or more photodetectors, wherein each of the multiple ring resonators is optically coupled to at least one of the one or more photodetectors; and at least a first microfluidic channel, wherein the first microfluidic channel fluidically couples the sensing ring resonator and an analyte delivery system.

2. The device of embodiment 1, wherein the first waveguide, the sensing ring resonator, the multiple ring resonators, the one or more photodetectors comprise an integrated photonic device.

3. The device of embodiment 2, wherein the integrated photonic device further comprises at least the first microfluidic channel.

4. The device of embodiment 1, wherein the multiple ring resonators are cascade coupled to the sensing ring resonator.

5. The device of embodiment 1, wherein the multiple ring resonators are optically coupled to a second waveguide which is optically coupled to the sensing ring resonator.

6. The device of embodiment 1, wherein each of the multiple ring resonators is optically coupled to a photodetector of a photodetector array.

7. The device of embodiment 1, wherein the sensing ring resonator is a subwavelength grating ring resonator.

8. The device of embodiment 1, wherein the multiple ring resonators are subwavelength grating ring resonators.

9. The device of embodiment 1, wherein the sensing ring resonator is a slot ring resonator.

10. The device of embodiment 1, wherein the multiple ring resonators are slot ring resonators.

11. The device of embodiment 1, wherein the sensing ring resonator and the multiple ring resonators comprise a self-compensated optical filter structure.

12. The device of embodiment 1, wherein the sensing ring resonator and the multiple ring resonators are of substantially similar radii.

13. The device of embodiment 1, wherein the sensing ring resonator and multiple ring resonator filters of different radii.

14. The device of embodiment 1, wherein the sensing ring resonator and the multiple ring resonators are of substantially similar cross-sectional area.

15. The device of embodiment 1, wherein the sensing ring resonator has a first radius, wherein multiple ring resonators have a range of radii, wherein the first radius lies within the range of radii.

16. The device of embodiment 1, wherein the sensing ring resonator has a first cross sectional area, wherein the multiple ring resonators have cross-sectional areas substantially similar to the first cross sectional area.

17. The device of embodiment 1, further comprising at least a second microfluidic channel, wherein the second microfluidic channel fluidically couples the multiple ring resonators and an analyte delivery fluid.

18. The device of embodiment 17, wherein the first microfluidic channel and the second microfluidic channel are fluidically coupled.

19. The device of embodiment 17, wherein the analyte delivery system comprises liquid-based analyte delivery system, wherein the liquid-based analyte delivery system delivers the analyte delivery fluid to at least the first microfluidic channel.

20. The device of embodiment 19, wherein the liquid-based analyte delivery system delivers the analyte delivery fluid to the second microfluidic channel.

21. The device of embodiment 19, wherein the analyte delivery system further comprises a gas-based analyte delivery system, wherein the gas-based analyte delivery system exposes the analyte delivery fluid to the analyte.

22. The device of embodiment 21, wherein the gas-based analyte delivery system exposes a portion of the analyte delivery fluid to the analyte and wherein the liquid-based analyte delivery system delivers the portion of the analyte delivery fluid which has been exposed to the analyte to the first microfluidic channel and analyte delivery fluid which has not been exposed to the analyte to the second microfluidic channel.

23. The device of embodiment 1, further comprising a pre-filtering stage, wherein the pre-filtering stage is optically coupled to the first waveguide.

24. The device of embodiment 23, wherein the pre-filtering stage comprises an arrayed waveguide grating.

25. The device of embodiment 23, wherein the pre-filtering stage comprises at least filtering ring resonator.

26. The device of embodiment 1, wherein an effective index of refraction of the sensing ring resonator changes when the sensing ring resonator is exposed to the analyte.

27. The device of embodiment 1, wherein the sensing ring resonator is functionalized so that a surface of the sensing ring resonator interacts with the analyte.

28. The device of embodiment 1, wherein the first waveguide, the sensing ring resonator, the multiple ring resonators, and the one or more photodetectors comprise a monolithically integrated photonic device.

29. The device of embodiment 1, wherein the first waveguide, the sensing ring resonator, and the multiple ring resonators comprise passive photonics.

30. The device of embodiment 29, wherein the passive photonics comprise silicon nitride devices.

31. The device of embodiment 1, wherein the one or more photodetectors and one or more optical source comprise active photonics.

32. The device of embodiment 31, wherein the active photonics comprise silicon devices.

33. The device of claim 1, further comprising a light source optically coupled to the first waveguide.

34. The device of claim 33, wherein the light source is a laser diode or light emitting diode.

35. The device of claim 1, further comprising at least a first element of a signal processing system.

36. The device of claim 1, further comprising a signal processor.

37. A method for forming a device as in any one of embodiments 1 to 36.

38. A method for operating a device as in any one of embodiments 1 to 36.

39. The method of embodiment 38, further comprising supplying an analyte to an analyte delivery system.

40. The method of embodiment 39, wherein supply the analyte to the analyte delivery system comprises a user breathing into a collection device fluidically coupled to the first microfluidic channel.

41. An analyte detection array, comprising: one or more sensor, wherein each sensor comprises: a waveguide; a sensing ring resonator, optically coupled to the waveguide, wherein the sensing ring resonator is sensitive to an analyte; multiple ring resonators optically coupled to the sensing ring resonator, photodetectors optically coupled to the multiple ring resonators; and a microfluidic channel, wherein the microfluidic channel fluidically couples the sensing ring resonator and an analyte delivery system.

42. The analyte detection array of embodiment 41, wherein each sensing ring resonator is sensitive to a different analyte.

43. The analyte detection array of embodiment 41, wherein each sensing ring resonator has substantially similar radii as the multiple ring resonators to which it is optically coupled.

44. The analyte detection array of embodiment 41, wherein each sensing ring resonator has a substantially similar cross-sectional area as the multiple ring resonators to which it is optically coupled.

45. The analyte detection array of embodiment 41, wherein the sensing ring resonators of the one or more sensors have substantially different radii.

46. The analyte detection array of embodiment 41, wherein the sensing ring resonators of the one or more sensors have substantially different cross-sectional areas.

47. A method for forming a device as in any one of the embodiments 41 to 46.

48. A method for operating a device as in any one of the embodiments 41 to 46.

49. A device for analyte detection, comprising: a first waveguide; a sensor ring resonator, optically coupled to the first waveguide, wherein the sensor ring resonator is sensitive to an analyte; multiple filter ring resonators optically coupled to the sensor ring resonator, one or more detectors, wherein each of the multiple filter ring resonators is optically coupled to at least one of the one or more detectors; and at least a first microfluidic channel, wherein the first microfluidic channel configured to fluidically deliver an analyte to the sensor ring resonator.

50. The device of embodiment 49, further comprising a microfluidic system, wherein the microfluidic system comprises the first microfluidic channel and a second microfluidic channel and wherein the multiple filter ring resonators are fluidically coupled to the second microfluidic channel.

51. The device of embodiment 49 or 50, wherein the sensor ring resonator and the multiple filter ring resonators are in fluidic communication with an analyte delivery fluid.

52. The device of any one of embodiments 49 to 51, further comprising an analyte delivery system fluidically coupled to the first microfluidic channel.

53. The device of any one of embodiments 49 to 52, further comprising a processor or memory communicatively coupled to the one or more detectors.

54. The device of embodiment 53, wherein the processor or memory is monolithically integrated into a substrate containing the sensor ring resonator.

55. The device of embodiment 53, wherein the processor or memory is heterogeneously integrated with a substrate containing the sensor ring resonator.

56. The device of any one of embodiments 49 to 55, further comprising an optical source, wherein the optical source is monolithically integrated into a substrate containing the sensor ring resonator.

57. The device of any one of embodiments 49 to 56, wherein the sensor ring resonator and the multiple filter ring resonators have substantially similar cross-sectional areas and different radii.

58. The device of any one of embodiment 49 to 57, wherein the sensor ring resonator operates as a filter for a first range of wavelengths and wherein each of the multiple filter ring resonators operates as a filter for a corresponding sub-range of wavelengths.

59. The device of any one of embodiments 49 to 58, wherein the first microfluidic channel is configured to receive an analyte in a first analyte delivery fluid and to deliver to analyte to the sensor ring resonator in a second analyte delivery fluid.

60. The device of any one of embodiments 49 to 59, wherein a surface of the sensor ring resonator is functionalized to interact with an analyte.

61. The device of any one of embodiments 49 to 60, wherein a refractive index of the sensor ring resonator is altered by interaction with the analyte.

62. The device of any one of embodiments 49 to 61, wherein the one or more detectors are configured to detect a change in the multiple filter ring resonators corresponding to interaction of the sensor ring resonator with one particle or more of analyte.

63. A method for operating a device as in any one of claims 49 to 62.

1. A device for analyte detection, comprising:
a first waveguide;
a sensor ring resonator, optically coupled to the first waveguide, wherein the sensor ring resonator is sensitive to an analyte;
multiple filter ring resonators optically coupled to the sensor ring resonator, one or more detectors, wherein each of the multiple filter ring resonators is optically coupled to at least one of the one or more detectors; and
at least a first microfluidic channel, wherein the first microfluidic channel configured to deliver an analyte-delivery fluid to the sensor ring resonator.

2. The device of claim 1, further comprising a microfluidic system, wherein the microfluidic system comprises the first microfluidic channel and a second microfluidic channel and wherein the multiple filter ring resonators are configured to fluidically couple to the second microfluidic channel.

3. The device of claim 1, wherein the sensor ring resonator and the multiple filter ring resonators are in fluidic communication with an analyte-delivery fluid.

4. The device of claim 1, further comprising an analyte delivery system, the analyte delivery system configured to fluidically couple to the first microfluidic channel.

5. The device of claim 1, further comprising a processor or memory communicatively coupled to the one or more detectors.

6. The device of claim 5, wherein the processor or memory is monolithically integrated into a substrate containing the sensor ring resonator.

7. The device of claim 5, wherein the processor or memory is heterogeneously integrated with a substrate containing the sensor ring resonator.

8. The device of claim 1, further comprising an optical source, wherein the optical source is monolithically integrated into a substrate containing the sensor ring resonator and wherein the optical source is configured to optically couple to the first waveguide.

9. The device of claim 1, wherein the sensor ring resonator and the multiple filter ring resonators have substantially similar cross-sectional areas and different radii.

10. The device of claim 1, wherein the sensor ring resonator operates as a filter for a first range of wavelengths and wherein each of the multiple filter ring resonators operates as a filter for a corresponding sub-range of wavelengths.

11. The device of claim 1, wherein the first microfluidic channel is configured to receive an analyte in a first analyte delivery fluid and to deliver to analyte to the sensor ring resonator in a second analyte delivery fluid.

12. The device of claim 1, wherein a surface of the sensor ring resonator is functionalized to interact with the analyte.

13. The device of claim 1, wherein a refractive index of the sensor ring resonator is altered by interaction with the analyte.

14. The device of claim 1, wherein the one or more detectors are configured to detect a change in the multiple filter ring resonators or output thereof corresponding to interaction of the sensor ring resonator with one particle or more of analyte.

15. A method for analyte detection comprising:
providing, via a first waveguide, optical input to a sensor ring resonator, wherein the sensor ring resonator optically coupled to the first waveguide and wherein the sensor ring resonator is sensitive to an analyte;
detecting, by one or more detectors, optical output from one or more of multiple filter ring resonators, wherein each of the multiple filter ring resonators optically coupled to the sensor ring resonator and wherein each of the multiple filter ring resonators is optically coupled to at least one of the one or more detectors; and
wherein a first microfluidic channel is configured to deliver an analyte-delivery fluid to the sensor ring resonator.

16. The method of claim 15, further comprising proving, by the first microfluidic channel, an analyte delivery fluid to the sensor ring resonator.

17. The method of claim 16, further comprising providing, by one or more additional microfluidic channels, the analyte delivery fluid to the multiple filter ring resonators.

18. The method of claim 15, further comprising determining, based on the optical output of the one or more of the multiple filter ring resonators, if a change in the optical output corresponds to an interaction of the sensor ring resonator with one or more particle of the analyte.

19. The method of claim 15, further comprising steps for determining if an analyte is present in the analyte-delivery fluid based on the detected optical output.

20. The device of claim 1, further comprising means for determining whether an analyte is present based on output of the one or more detectors.
